(19) United States
(12) Patent Application Publication
(10) Pub. No.: US 2011/0053257 A1
(43) Pub. Date: Mar. 3, 2011

(54) Photo-bioreactor with Particle Separation and Water Recovery System

(76) Inventors: Ian Lane Ragsdale, San Diego, CA (US); Oscar Luna, Chula Vista, CA (US)

(21) Appl. No.: 12/860,622

(22) Filed: Aug. 20, 2010

Related U.S. Application Data
(60) Provisional application No. 61/274,762, filed on Aug. 21, 2009.

(51) Int. Cl. C12M 1/00

(52) U.S. Cl. 435/292.1

(57) ABSTRACT

The Photo-bioreactor with Particle Concentration and Water Recovery System is a bioreactor system designed to propagate algae under fully closed, controlled conditions. The system is highly efficient and has a reduced turnaround time compared to similar photo-bioreactors. The system has an integrated, gravity-flow biomass harvest and water recovery system that allows the operator to simultaneously harvest the cell concentrate and recycle the water used in the growth media. The bioreactor's scale can range from a single bench-top unit to multiple, large-scale commercial units in a manifold type array, and can utilize sunlight or artificial light.

### CROSS-REFERENCE TO RELATED APPLICATIONS

The inventors have previously filed the following related provisional patent applications:

Panelized Solar Bioreactor System for Growth of Photosynthetic Microorganisms

Application Number: 61/274,762

Filing Date: Aug. 21, 2009

Particle Separation and Water Recovery System for Panelized Solar Bioreactor

Application Number: Unknown

Filing Date: Unknown, Sent to USPTO on Aug. 17, 2010 via Certified Mail#7009 3410 0002 4328 8146

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The photo-bioreactor can be used for both bench-scale and large-scale-commercial cultivation of algae biomass. Current and future applications for such a device include production of bio-diesel, cellulosic ethanol, feed for livestock and commercially grown aquatic animals and coral, nutri-ceuticals, and nutritional supplements.

There are several bioreactor designs currently available for use, however a few factors are of particular concern regarding their large-scale commercial use. One major concern is cost of production and maintenance. Another issue is fouling of transparent surfaces due to calcium and other deposits produced during algae cultivation. Harvesting and separation generally require additional equipment and energy expenditures as well.

### BRIEF SUMMARY OF THE INVENTION

The Photo-bioreactor with Particle Separation and Water Recovery System can be used as a stand-alone unit for small to moderate scale algae production or research purposes and can also be configured as manifold-type array with multiple, identical units for large scale commercial applications. The focus of the invention was to create a system that can be produced at relatively low costs, while facilitating ease of maintenance and low energy consumption. While the unit is designed to utilize solar energy, artificial light sources can be used as well for research purposes.

The reactor has no moving mechanical parts, and uses air bubbles as the primary mode of mixing and pH control. The micro-bubbles travel vertically through the reactor in a swirling action to produce agitation when necessary. The air pumps can be cycled on or off to control the pH within the system. In general, atmospheric air will be filtered and pumped directly through the bubble generator, but CO2 supplementation can be used when necessary. As is common for reactor designs, probes for temperature, pH, and dissolved gas monitoring can be used as well.

The acrylic sheet material used in its construction was chosen for its low cost, strength and ability to withstand the elements. As this material is used in the construction of many medium and large sized aquariums, methods of construction are well known to most aquarium builders. Due to its simple design, the reactor is inexpensive and easy to reproduce. The incorporation of removable panels and subsections allows for cleaning to remove fouling and buildup on the reactor's transparent surfaces, while magnetic aquarium brushes can be used to keep surfaces clean while in use. The particle concentration chamber incorporated in the design utilizes gravity as the driving force for biomass separation and harvest, providing a considerable reduction in energy costs over mechanical separation methods. The filtration membrane can be easily replaced when the reactor is empty. Furthermore, 95-99+% of the water is recycled in a single step. This reduces the cost of production as well as down time between production runs.

### BRIEF DESCRIPTION OF THE SEVERAL VIEWS OF THE DRAWING

FIG. 1 Growth Chamber

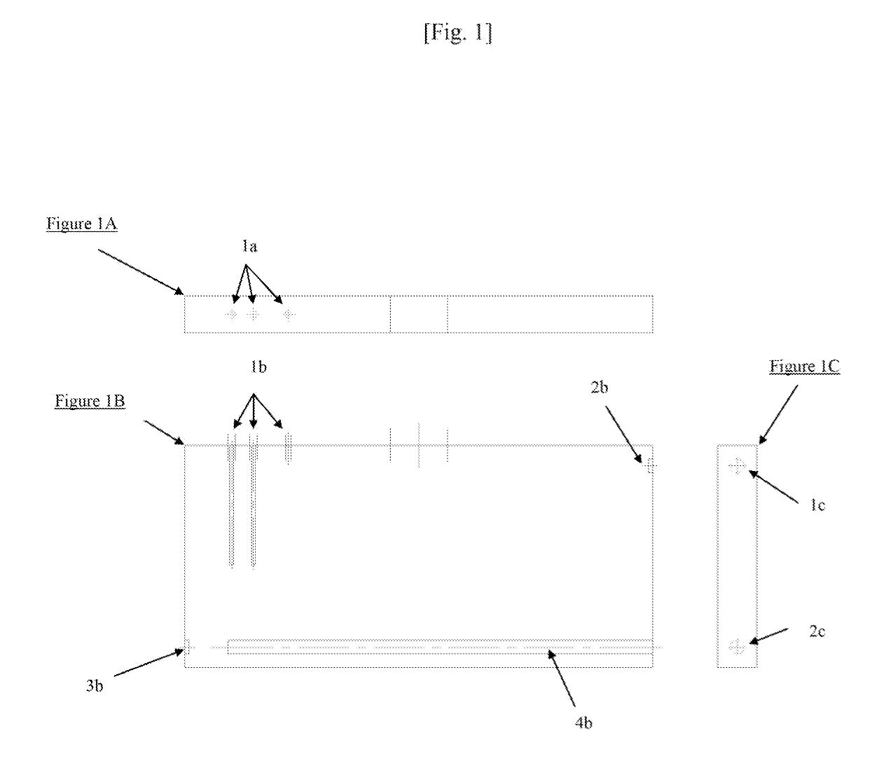

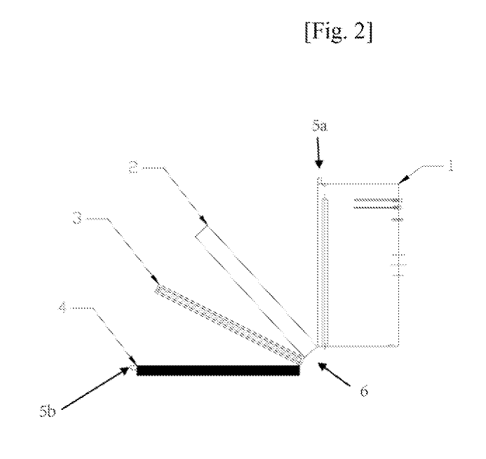

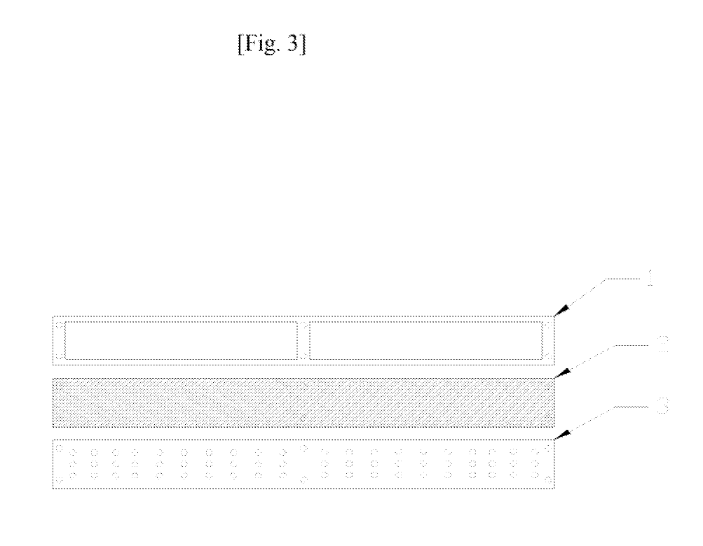

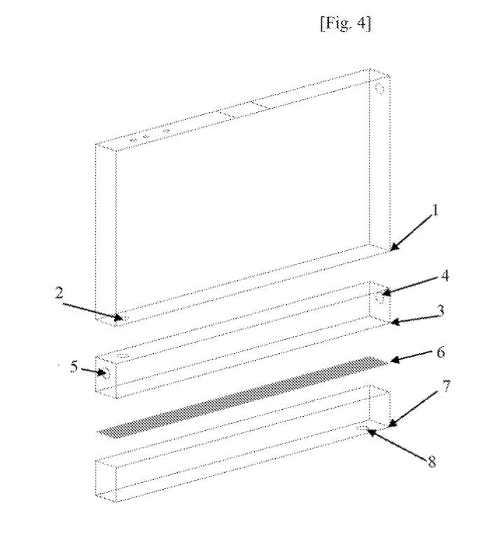

FIG. 1A: Top View
1a. Ports for environmental monitoring probes. pH, temp. and dissolved gasses.

FIG. 1B: Side View
1b. Ports for environmental monitoring probes. pH, temp. and dissolved gasses.
2b. Media inlet valve
3b. Media outlet valve
4b. Bubble Generator

FIG 1C: End View
1c. Media inlet valve (can be placed on either side)
2c. Media outlet valve (can be placed on either side)

FIG. 2
1. Growth chamber (not shown to scale)
2. Concentration chamber (bolts to FIG. 2.1, separated by stage 1 drain valve)
3. Three-layer filtration membrane sandwich (comprised of two rigid sheets with a porous membrane in between, and gaskets around the outer rim)
4. Water recovery chamber (comprised of inset channel for filtration membrane and stage 2 drain valve)
5a. upper clamp
5b. lower clamp
6. Triple hinge

FIG. 3
1. Upper section—rigid, open in center
2. Center section—filtration membrane
3. Lower section—rigid, perforated

FIG. 4
1. Growth
2. Phase 1 drain valve
3. Particle concentration chamber
4. Cross-flow inlet valve
5. Cross-flow outlet valve
6. Three-layer replaceable filtration membrane sandwich
7. Water recovery chamber
8. Phase 2 drain valve

### DETAILED DESCRIPTION OF THE INVENTION

The Photo-bioreactor System is designed to use sunlight and or artificial light to propagate multiple types of photosynthetic aquatic microorganisms for the purpose of biomass recovery through filtration and concentration. The photo-bioreactor consists of a hollow, transparent panel that is constructed of clear acrylic sheet material of a thickness proportional to the respective media weight. It comprises of multiple ports for the addition of media, O2, CO2, and environmental monitoring probes. The reactor is comprised of four distinct parts including the following; the reactor, a concentration chamber, a filtration membrane and a water recovery chamber. The concentration chamber, located beneath the growth chamber and connected to growth chamber via an electronic or mechanically controlled drain valve, sits atop a porous filter-membrane that permeates particles below the filter's pore size for the purpose of concentrating the biomass. The filtration membrane is comprised of three parts; a replaceable filter membrane sandwiched between two rigid panels with outer gaskets, the upper section being open and the lower section being perforated to allow flow-through, while structurally supporting the filter membrane. The filtrate is directed towards the water recovery chamber, which has an additional electronic or mechanically controlled drain valve at its lowest point. The orientation of each of the four sections is such that fluid can travel from the uppermost to lowermost chamber using only gravitational force. Furthermore, all four sections are to be connected at one end using a hinge system that will allow for access to filtration membrane and lower chambers, as well as a removable side panel for periodic cleaning.

The reactor uses atmospheric air or exhaust, which is bubbled vertically through the media contained within the panel, to provide a carbon source for the algae growing in the media. Air holes are oriented in a fashion that facilitates swirling action to maximize mixing. The bubbles keep the algae suspended evenly within the media, which allows maximum exposure to sunlight. Furthermore, the bubble environment can be cycled on and off in order to control temperature, pH, and dissolved gasses. Further temperature control could be achieved through a solar heat exchanger system.

Full-scale reactors, for use in scale-up production, may be arranged in manifolds of 20-100 reactors having a capacity of 500-1400 Liters each reactor. The manifolds will have a centralized control system to deliver media, control effluent gasses, and facilitate biomass harvest. The manifold-type array system for the large scale bioreactors is designed to allow for a central processing and control module for multiple reactors. The control module would monitor temperature, pH, dissolved gasses, exhaust gasses, and allow for sampling of each independent system. It would include an auxiliary tank for mixing media and harvesting biomass that is placed below ground to facilitate gravity-flow filtration during harvest.

Small-scale reactors for the bench top may use artificial light sources and/or reactor controllers and have a capacity of approximately 10-100 Liters. The purpose of the smaller reactor size is to allow for research or testing before introducing a culture into a large scale reactor. These small reactors may also be utilized for other applications that do not require large amounts of biomass. The small scale system uses pH and % CO2 probes to monitor the internal environment, and has been tested using a New Brunswick BioFlo reactor controller, but can be utilized as a stand-alone device as well.

1. A transparent photo-bioreactor comprised of four distinct sections;
the uppermost section being the growth chamber, through which atmospheric air or exhaust can be bubbled vertically to aerate and mix the growth media, and having probes for environmental monitoring and inlet and outlet valves for recirculation of media as well as an electronic or mechanically controlled drain valve leading into the concentration chamber beneath the growth chamber;
a concentration chamber having a volume fractionally and complementarily proportionate to the volume of water which is to be recycled during harvest, being directly beneath the growth chamber connected through a drain valve and having at least two horizontally placed valves for cross-flow removal of concentrated material with an open bottom connecting to the filtration membrane;
a filtration membrane comprised of a replaceable filter sandwiched between two sections of rigid panel with outer gaskets, the upper section being open and the lower section having a perforated matrix for passage of filtrate into the water recovery chamber;
a water recovery chamber with inset channel to accept filtration membrane and an electronic or mechanically controlled drain valve at its lowest point for passage of filtrate out of the system.

2. The combination defined in claim 1, wherein the shape of the reactor and subsequent lower chambers are in cylindrical or square in shape.

3. A manifold-type array wherein bioreactors described in claim 1 are connected either parallel or in series, with multiple reactors connected to a central control unit.
